(19) United States
(12) Patent Application Publication
(10) Pub. No.: US 2016/0304861 A1
(43) Pub. Date: Oct. 20, 2016

(54) SPATIAL MULTIPLEXING FOR MULTISIGNAL CELLULAR IMAGING

(71) Applicant: Massachusetts Institute of Technology, Cambridge, MA (US)

(72) Inventors: Guangyu Xu, Amherst, MA (US); Edward Boyden, Chestnut Hill, MA (US); Kiryl D. Piatkevich, Cambridge, MA (US); Katarzyna Adamala, Cambridge, MA (US)

(73) Assignee: Massachusetts Institute of Technology, Cambridge, MA (US)

(21) Appl. No.: 15/099,232

(22) Filed: Apr. 14, 2016

Related U.S. Application Data
(60) Provisional application No. 62/147,225, filed on Apr. 14, 2015.

(51) Int. Cl. C12N 15/10

(52) U.S. Cl. CPC C12N 15/1086

(57) ABSTRACT

The invention, in some aspects, relates to methods, systems, and components of a high-content, single-cell resolution, spatial multiplex cell imaging system.

### RELATED APPLICATIONS

This application claims benefit under 35 U.S.C. §119(e) of U.S. Provisional application Ser. No. 62/147,225 filed Apr. 14, 2015, the disclosure of which is incorporated by reference herein in its entirety.

### GOVERNMENT INTEREST

This invention was made with government support under the NSF CBET 1344219 grant awarded by the National Science Foundation. The Government has certain rights in the invention.

### FIELD OF THE INVENTION

The invention, in some aspects, relates to methods, systems, and components of a high-content, single-cell resolution, spatial multiplex cell imaging system.

### BACKGROUND OF THE INVENTION

Currently it is not possible to monitor more than one or two signals in a living cell at the same time. The difficulty, is at least in part because the spectra of fluorescent biological reporters are limited to a few colors, e.g. emitting in green or red, which compromises the successful use of imaging methods in living cells.

### SUMMARY OF THE INVENTION

According to one aspect of the invention, methods of spatial multiplex imaging in a cell are provided. The methods including: expressing in a cell one, two, or more effector compounds each comprising: (a) an independently selected sensor molecule comprising an indicator molecule and (b) an independently selected effector molecule, wherein the effector molecule binds a predetermined anchor site in the cell, and when two or more effector molecules are expressed in the cell each binds a different predetermined anchor site in the cell; and wherein the sensor molecule comprises an independently selected indicator molecule; and detecting the one, two or more independently selected indicator molecules in the cell. In certain embodiments, two or more effector compounds are expressed in the cell, and detecting comprises detecting each of the independently selected indicator molecules. In certain embodiments, the method also includes determining a cellular activity or signal indicated by the detection of the indicator molecule of the independently selected sensor molecules of the one, two, or more effector compounds. In some embodiments, the anchor site comprises chromosomal DNA. In some embodiments, the indicator molecule comprises a fluorescent polypeptide. In certain embodiments, the effector compound comprises a TALE polypeptide and sensor molecule comprising a fluorescent polypeptide indicator. In some embodiments, the cell is a mammalian cell. In some embodiments, the number effector compounds expressed in the cell is between 2 and 100. In certain embodiments, the anchor site comprises a sequence of chromosomal DNA that specifically binds the effector molecule, the binding of the effector molecule is chromosome specific; and wherein the chromosomal DNA that specifically binds the effector molecule is present in no more than one chromosome of the cell and contains at least 5 consecutive repeats of the specific DNA sequence to which the effector molecule binds. In some embodiments, the anchor site comprises a sequence of chromosomal DNA that specifically binds the effector molecule is present in no more than one chromosome of the cell, contains at least 6, 7, 8, 9, 10, 11, 12, 13, 14, 15, 20, 30, 40, 50 consecutive repeats of the specific DNA sequence to which the effector molecule binds, and contains no active genes. In some embodiments, the sensor molecule is independently selected from GCaMP6f, GEICS1.3, AKAR4, and ICUE3.

According to another aspect of the invention, methods of identifying a spatial multiplex imaging binding site are provided. The methods include: (a) selecting a first effector compound comprising a first sensor molecule comprising a first indicator molecule and a first effector molecule that specifically binds a predetermined anchor site in a cell, wherein the anchor site comprises a sequence of chromosomal DNA that specifically binds the first effector molecule, the binding of the first effector molecule is chromosome specific; and the chromosomal DNA that specifically binds the first effector molecule is present in not more than one chromosome of the cell, and contains at least 5 consecutive repeats of the specific DNA sequence to which the first effector molecule binds; (b) expressing in a cell the first effector compound; and (c) detecting the first indicator molecule that is expressed in the cell. In some embodiments, the method also includes: (d) selecting one or more additional independently selected effector compounds each comprising: (i) an independently selected sensor molecule comprising an independently selected indicator molecule and (ii) an independently selected effector molecule that specifically binds an anchor site in a cell, wherein the anchor site comprises a sequence of chromosomal DNA that specifically binds the independently selected effector molecule, the binding of the independently selected effector molecule is chromosome specific; and the chromosomal DNA that specifically binds the independently selected effector molecule is: present in not more than one chromosome of the cell, and contains at least five consecutive repeats of the specific DNA sequence to which the independently selected effector molecule binds; (e) expressing in the cell of (b) the one or more additional independently selected effector compounds; and (f) detecting the indicator molecule of the first effector compound and the indicator molecule of the one or more independently selected effector compounds expressed in the cell. In certain embodiments, the anchor site comprises a sequence of chromosomal DNA that specifically binds the effector molecule, the binding of the effector molecule is chromosome specific; and wherein the chromosomal DNA that specifically binds the effector molecule is present in one chromosome of the cell, contains at least 2, 3, 4, 5, 6, 7, 8, 9, 10, 11, 12, 13, 14, 15, 20, 30, 40, or 50 consecutive repeats of the specific DNA sequence to which the effector molecule binds, and contains no active genes. In some embodiments, the indicator molecule is a fluorescent indicator polypeptide. In certain embodiments, the effector compound comprises a TALE polypeptide and sensor molecule comprising a fluorescent indicator molecule. In some embodiments, the effector compound comprises a TALE polypeptide and sensor molecule comprising a fluorescent polypeptide indicator. In some embodiments, the cell is a mammalian cell. In certain embodiments, the sensor molecule is independently selected from GCaMP6f, GEICS1.3, AKAR4, and ICUE3.

According to yet another aspect of the invention, effector compounds are provided. The effector compounds include (a) a sensor molecule comprising an indicator molecule and (b) an effector molecule that when expressed in a cell, comprises a DNA-binding sequence that binds a predetermined chromosomal DNA anchor site in the cell, wherein the binding of the expressed effector molecule is chromosome specific; and wherein the chromosomal DNA that specifically binds the expressed effector molecule is present in no more than one chromosome type in the cell, contains at least 5, 6, 7, 8, 9, 10, 11, 12, 13, 14, 15, 20, 30, 40, or 50 consecutive repeats of the specific DNA sequence to which the expressed effector molecule binds, and contains no active genes. In some embodiments, the effector compound is in a composition comprising a carrier, optionally a pharmaceutically acceptable carrier. In some embodiments, the effector compound is a nucleic acid molecule. In certain embodiments, the nucleic acid molecule is in a vector. In some embodiments, the effector compound is a polypeptide molecule. In some embodiments, the polypeptide is a fusion protein.

According to another aspect of the invention, vectors are provided. The vectors include one, two, or more polynucleotide sequences each encoding an independently selected effector compound, wherein each effector compound comprises: (a) an independently selected sensor polypeptide comprising an independently selected indicator polypeptide and (b) an independently selected effector polypeptide that specifically binds a predetermined anchor site when expressed in a cell. In some embodiments, the number effector compounds encoded in the vector is between two and five. In certain embodiments, the anchor site comprises a sequence of chromosomal DNA that specifically binds the effector molecule, the binding of the effector molecule is chromosome specific; and the chromosomal DNA that specifically binds the effector molecule is present in no more than one chromosome of the cell, contains at least 5 consecutive repeats of the specific DNA sequence to which the effector molecule binds, and contains no active genes. In some embodiments, the encoded independently selected indicator molecule is a fluorescent polypeptide. In some embodiments, two or more of the expressed independently selected indicator molecules are different from each other. In some embodiments, two or more of the expressed independently selected indicator molecules are the same as each other. In certain embodiments, the indicator molecule comprises a fluorescent polypeptide. In some embodiments, the effector compound comprises a TALE polypeptide and sensor molecule comprising a fluorescent polypeptide indicator. In some embodiments, the sensor molecule is independently selected from GCaMP6f, GEICS1.3, AKAR4, and ICUE3. In certain embodiments, the vector is in a composition. In some embodiments, the composition further comprises a carrier, optionally a pharmaceutically acceptable carrier. In certain embodiments, the vector is in a cell. In some embodiments, the cell is a mammalian cell. In some embodiments, the cell is an in vitro cell.

According to yet another aspect of the invention, methods for a spatial multiplex imaging chromosome anchor site are provided. The methods including: determining a TALE chromosome binding site having a polynucleotide sequence that (a) includes more than 20 repetitive chromosome binding sequences, (b) the binding site sequence is between 18 and 22 by in length, and (c) optionally begins with the polynucleotide thymine; wherein the determined TALE chromosome binding site is identified as a spatial multiplex imaging chromosome anchor site. In certain embodiments, the chromosome is a mammalian chromosome. In some embodiments, the method also includes confirming that the identified anchor site is at least one of: chromosome specific and at least 5000 by away from an active gene. In certain embodiments, each of the TALE chromosome binding site polynucleotide sequence includes 20 repetitive chromosome binding sequences, each of the binding sequences is 20 by in length, and the first nucleotide of the sequences is a thymine.

### BRIEF DESCRIPTION OF THE DRAWINGS

FIG. 1A-C shows results from HEK293 cell experiments indicating sensor clustering and typical responses to an ionomycin treatment in HEK cells. FIG. 1A shows Gcamp6f sensor clustering (green). FIG. 1B shows an overlay image of the bright field (grey) and the Gcamp sensor clustering. Both images were taken with with 100×, 1.45 NA. Scale bar: 6 um. FIG. 1C shows how the Gcamp6f sensor cluster responded to an ionomycin treatment to the cell. The fluorescence values were normalized after subtracting the extracellular background.

FIG. 2A-D shows DNA FISH results in HEK cells from experiments performed to assure the binding specificity of TALE proteins. FIG. 2A shows GCaMP6f sensors (green); FIG. 2B shows DNA FISH probes (red); FIG. 2C shows NucBlue nuclear stain; FIG. 2D shows an overlay image of FIG. 2A, FIG. 2B, and FIG. 2C. All images were taken with 20×, 0.45 NA. Scale bar: 12 μm.

FIG. 3A-D shows results of Hippocampus mouse neuron experiments. The images show typical sensor clustering and responses in hippocampus mouse neurons. FIG. 3A shows one neuron single-transfected with Gcamp6f sensors. Typically two sensor clusters (green) were expressed in the cell (bright field in grey); FIG. 3B shows results demonstrating that a typical neuron single-transfected with Gcamp6f, all clusters show identical fluorescence patterns as neuron spikes; FIG. 3C shows results of one neuron co-transfected with Gcamp6f and GEICS 1.3 sensors. Typically three sensor clusters (green) were expressed in the cell (bright field in grey); FIG. 3D shows results demonstrating that in a typical neuron co-transfected with Gcamp6f and GEICS 1.3, sensor clusters showed different fluorescence patterns as neuron spikes; Images in FIG. 3A and FIG. 3C were imaged with 100× 1.45 NA. Scale bar: 12 μm. The fluorescence values in FIG. 3B and FIG. 3D were normalized after subtracting the background in cell nucleus.

FIG. 4A-C shows photomicroscopy images and graphs illustrating results obtained in MEF cells that expressed embodiments of FRET sensors. The results showed typical FRET sensor clustering and responses in mouse embryonic fibroblast (MEF) cells. FIG. 4A shows results from one MEF cell single-transfected with AKAR4 sensors with YFP/CFP FRET pairs. Sensor clusters were expressed in the cell, confirmed by the co-localization between YFP and CFP channels (left panel); a 50 μM FSK stimulus (added at around 2 min after start of recording) caused consistent increase of the fluorescence ratio, YFP/CFP. FIG. 4B shows results from another MEF cell single-transfected with ICUE3 sensors with YFP/CFP FRET pairs; a 50 μM FSK stimulus caused consistent decrease of the fluorescence ratio, YFP/CFP. FIG. 4C shows results from another MEF cell that was co-transfected with both AKAR4 and ICUE3 sensors with YFP/CFP FRET pairs; a 50 μM FSK stimulus caused a consistent increase of the fluorescence ratio YFP/CFP for AKAR4 and caused a consistent decrease of the fluorescence ratio YFP/CFP for ICUE3. Images were imaged with 20× 0.75 NA. Scale bar: 30 μm. The fluorescence values were normalized after subtracting the extracellular background.

### DETAILED DESCRIPTION

The invention in some aspects relates to novel methods of spatial multiplex imaging in live cells. Imaging methods of the invention permit simultaneous imaging of 1, 2, 3, 4 or more signals in a living cell. Aspects of the invention include methods that permit spatially multiplexing reporter molecules, which can be used to simultaneously image one signaling state, or at least 2, 3, 4, 5, 6, 7, 8, 9, 10, 20, 40, 50, 100, or more signaling states simultaneously. The invention includes, in some embodiments, a high-content, single-cell resolution cell imaging system and methods for its use. In certain embodiments, methods of the invention can provide up to 10-20 multiplexed signal recordings in cells, and using only single or a few indicator signal polypeptides, for example though not intended to be limiting, only a single or a few colored genetically encoded fluorescent sensors. As used herein the terms sensor and reporter may be used interchangeably. The ability to use one or just a few colored genetically encoded fluorescent sensors allows high-content imaging with an epi-fluorescent microscope commonly used in laboratories. In some embodiments, the system and methods of the invention are based in part on the placement of spatial multiplexing sensors of interest in a cell nucleus, which allows simultaneous monitoring different cellular signals indicated by indicator sensors or signals that are anchored in chromosome DNAs in a spatially resolvable manner. In some embodiments of the invention the indicator signals are fluorescent indicator signals. Some embodiments of the invention include methods in which sensors of interest that are fused to effector molecules are expressed in cells. In certain aspects of the invention, an effector molecule is a DNA binding protein. A non-limiting example of a method of expressing a sensor/signal polypeptide of interest for fusion to DNA binding proteins comprises use of transcription activator-like effectors (TALE). Transcription activator-like effectors (TALEs) are highly specific, high-affinity DNA-binding proteins that can be fused to other molecules, for example a sensor or indicator molecule, such as, but not limited to a fluorescent indicator molecule. Together, the TALE and the sensor molecule can be expressed in a cell and can target and bind to nearly any site in a genome. In certain embodiments of the spatial multiplex imaging methods of the invention include effector compounds that include one or more TALE compounds that comprise an indicator signal molecule, (a non-limiting example of which is a fluorescent indicator molecule). When expressed in a cell, such an effector compound results in clustering of the indicators due to the localized binding of the TALE compounds in the cell. For example, in certain aspects of the invention, the clustered indicators (sensors) are localized in the cell nucleus with spatial separations larger than the diffraction limit of a microscope used to image the cell, which means that when two or more different effector compounds are expressed in one or more cells, each different indicator reporting corresponding to nuclear signals to be imaged independently from each other.

In certain aspects of the invention, methods include identifying a set of binding sites that meet three criteria: 1) binding sites that are chromosome specific, with each binding site exists in one chromosome only, maximizing the chance of getting spatially resolvable binding sites; 2) binding sites that contain repetitive DNA sequences in a row, such that each sequence binds with one TALE array fused to a single sensor, allowing tens of sensors to cluster, gaining enough brightness to be imaged; and 3) binding sites that contain no active genes to lower probability of clustered indicators/sensors repressing gene expression in a cell. Bioinformatics analysis has been used to identify more than 20 such binding sites available in the human genome and more than 10 such binding sites in mouse genome, offering unprecedented multiplicity in single-cell fluorescence imaging. These binding sites can be used in methods and systems of the invention to multiplex any type of genetically encoded sensors, non-limiting examples of which are ionic or kinase sensors, either based on intensiometric or FRET readouts, within a single enabling simultaneous monitoring of a variety of cellular signals that can report, for instance, the gene regulation activities.

Monitoring cellular signals using methods of the invention can be used to report activities in the monitored cell or cells. A non-limiting example of an activity that may be monitored using methods of the invention is gene regulation activity. Methods of the invention have been utilized in HEK293 cells, primary hippocampus mouse neurons, and mouse embryonic fibroblast cells, demonstrating that methods of the invention have broad applicability in different cell lines.

The invention includes, in part, methods of spatial multiplexing, using genomic or other enduring anchor sites in at least one cell, to achieve multiplexed signaling readout from the at least one cell. In certain embodiments of the invention, TALE effectors coupled to fluorescent signaling/sensor indicator proteins may be used in methods of the invention to multiplex the reporting of cellular signaling and states. Another aspect of the invention includes methods of identifying one or more sets of binding sites that allow different types of sensors to cluster at different sites in a cell, (a non-limiting example of which is the nucleus of a cell), while not altering the gene expression or other function or process in the cell. The invention includes, in part, use of methods and systems for high-content cellular analysis, drug development or screening, and evaluations on treatment on diseases. Clinical applications for embodiments of methods and systems of the invention include, but are not limited to high-content cell status examination for drug screening and other treatment evaluation.

The invention, in part, relates to methods for spatial multiplexing using detectable labels (also referred to herein as “reporters” and “indicators”) that can be used to image multiple signaling states simultaneously, because the identity of each signaling molecule is encoded by its spatial position, (easily mapped using DNA hybridization techniques), even if the reporters have the same spectrum. Methods of the invention in some embodiments bring spatial multiplexing into biology, and open up the ability to image many signals at once in living cells and organisms using a spatial multiplexing system of the invention, which targets indicator signal polypeptide sensors that indicate different signaling states, to different sites in a cell. Various indicator detectable labels that may be used in methods of the invention include but are not limited to: fluorescent detectable labels, and which in some aspects of the invention comprise a polypeptide.

In some aspects of the invention, methods include spatial multiplex methods that target fluorescent reporters that optically indicate different cell signaling states, to different sites in a cell. This aspect of the invention is based, in part, on the fact that the genome contains just one copy of many different sequences, and thus a DNA binding polypeptide that targets these sequences can be used to localize a sensor at a unique site in the cell. Using methods of the invention, the DNA sequence to which a binding polypeptide is anchored is mapped, allowing identification of the signaling or activity that is being reported by the sensor molecule, with which the binding polypeptide was expressed and is attached. Methods of the invention, in some embodiments allow identification of the signal being reported via a sensor molecule, even if they include the same indicator molecules, as a non-limiting example, even if they include fluorescent indicator signal polypeptides that all emit the same color of light.

### Effector Molecules, Compounds, and Variants

An effector compound of the invention comprises a genetically encoded sensor molecule that includes an indicator molecule, and also includes a genetically encoded effector molecule. Each of the components of the effector molecule, referred to herein as: the sensor molecule, the indicator molecule, and the effector molecule, can be independently selected. As used herein the term “independently selected” in reference to sensor molecules, indicator molecules, and effector molecules means each of the components may be chosen for inclusion and included in effector compound of the invention separately and independently from other of the components used in a compound of method of the invention. For example, though not intended to be limiting, in an effector compound of the invention having an indicator that includes one type of fluorophore may be expressed in a cell with another effector compound that has an indicator that includes the same type or that includes a different type of fluorophore. Each component of an effector compound may be selected independently from other components of effector compounds.

Methods of the invention, in some aspects include expressing one, two, three, four, five, six, seven, eight, or more effector compounds in a cell. Each of the effector compounds may include (a) an independently selected sensor molecule comprising an indicator molecule and (b) an independently selected effector molecule. An effector molecule may be chosen because of its function of binding a predetermined anchor site in the cell in which the effector compound is expressed. In certain aspects of the invention, multiple effector molecules may be expressed in the cell and each effector molecule may bind a different predetermined anchor site in the cell than is bound by one or more of the other effector molecules. In some aspects of the invention, each of the effector molecules may bind a different predetermined anchor site in a cell than all of the other effector molecules expressed in the cell. Methods of the invention include, in some aspects, imaging and detecting one, two, three, four, five, or more independently selected indicator molecules that are expressed in a cell.

### Sensor Molecules

Methods of the invention may also include determining a cellular activity or signal in a cell in which an effector compound of the invention has been expressed. Detection of an indicator molecule permits determination of any change in the indicator signal, which indicates a change in a cellular activity or signal, as detected by the sensor molecule. In certain aspects of the invention, a sensor molecule may be selected to detect or monitor an activity or signal in a cell in which the effector compound, which includes the sensor molecule, is expressed. For example, though not intended to be limiting, a calcium or ion flux detecting sensor may be utilized in an effector compound of the invention and the indicator signals calcium or ion flux detected by the sensor in the cell. Non-limiting examples of genetically encoded sensors that may be used in compounds and methods of the invention are: ionic and kinase sensors. Non-limiting examples of sensor molecules that may be included in compounds and methods of the invention are: GCaMP6f, GEICS1.3, AKAR4, and ICUE3 molecules. It will be understood that imaging or visualization of an indicator polypeptide expressed in a cell corresponds to a physiological aspect of, alteration in, change of process in, and/or activity in the cell for which the sensor polypeptide is selected. For example, to detect in a cell aspects, alternations, or activities such as: gene regulation, pH change, calcium flux, etc., a sensor polypeptide that functions to detect one or more of such aspects, alternations, and activities and that signals the aspects, alterations, and activities via its attached indicator polypeptide, can be included in an effector compound of the invention and expressed in the cell. Non-limiting examples of sensors that can be included in compounds and methods of the invention are calcium flux sensors such as, but not limited to GCaMP6f, which can be used to detect calcium flux; AKAR4, which can be used to detect PKA kinase; CKAR, which can be used to detect PKC kinases; ICUE3, which can be used to detect cAMP changes; and small molecule sensors, a non-limiting example of which is: GEPRA, which can be used to detect retinoic acid. See for example: Depry, C., et al., 2011 Mol. BioSyst., 7, 52-58; Violin, J., et al., 2003 J. Cell Biology, 161,899-909; DiPilato, L. M. & J. Zhang Mol. BioSyst., 2009, 5, 832-837; and Shimozono, S. et al., 2013 Nature 496, 363-366, the content of each of which is incorporated herein by reference.)

In some embodiments of the invention, a genetically encoded sensor molecule includes an indicator molecule, also referred to herein as a detectable label. An indicator molecule may in some aspects of the invention be a fluorescent molecule. A non-limiting example of a fluorescent molecule that may be used in embodiments of the invention is a Förster resonance energy transfer (i.e. FRET) indicator. FRET-based sensors that that can be include in compounds of the invention and used in methods of the invention are known in the art.

### Effector Molecules

Another component of an effector compound of the invention is an effector molecule. In aspects or the invention, an effector molecule binds to a predetermined anchor site in the cell. In some aspects of the invention, an anchor site may comprise a sequence of chromosomal DNA to which the effector molecule specifically binds. In certain aspects of the invention, binding of the effector is chromosome specific and the chromosomal DNA that specifically binds the effector molecule is present in no more than one chromosome of the cell in which the effector compound is expressed. In certain aspects of the invention, the anchor site is a chromosomal DNA anchor site and the site includes no active genes. In certain aspects of the invention, an anchor site is at least 5000 by away from an active gene. Binding anchors that may useful in certain methods of the invention are DNA molecules that include consecutive repeats of the DNA sequence to which the effector molecule specifically binds. The length of a binding anchor repeat sequence may be at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, or 50 nucleic acids in length. The number of repeats of binding anchor sequences may be at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, 100 repeats in a row. The number of repeats results in a number of expressed effector polypeptides binding to their predetermined anchor region. Multiple repeats of the predetermined anchor sequence can increase the number of expressed effector polypeptides that bind to the predetermined anchor site, which amplifies the signal of the sensor's indicator that can be detected and imaged. In some embodiments of the invention, each of the TALE chromosome binding site polynucleotide sequences includes 20 repetitive chromosome binding sequences, each of the binding sequences is 20 by in length, and the first nucleotide of the sequences is a thymine.

As used herein, a predetermined site is a site that has been selected and binding sequences have been included in the effector molecule in order to selectively bind and anchor the expressed effector compound to the site in the cell in which it is expressed. In certain aspects of the invention, two or more effector molecules may be expressed in a cell and each may bind a different predetermined anchor site in the cell. Examples of anchor sites include, but are not limited to DNA anchor sites and chromosomal anchor sites. Examples of effector molecules that bind to predetermined anchor sites, although not intended to be limiting are Transcription activator-like effectors (TALE) molecules. TALE molecules are highly specific, high-affinity DNA-binding proteins that can be fused to other molecules. The binding location of TALE polypeptides may be predetermined. In certain embodiments of the invention, inclusion of a TALE polypeptide in an effector compound of the invention that is expressed in a cell, results in the TALE polypeptide binding to the predetermined anchor site, which indicates the specific location of cellular activity or changes that are detected in the cell by the sensor molecule that is expressed as part of the effector compound. In certain aspects of compounds and methods of the invention, an effector compound includes one or more TALE polypeptides and a sensor molecule comprising a fluorescent polypeptide indicator.

Because of the predetermined anchor site, the location of the expressed effector compound in a cell is known and the activity detected by the sensor molecule can be correlated with the anchor position in the cell. In certain aspects of the invention, detecting an indicator expressed in an effector compound of the invention provides information and permits monitoring of an activity or signal in the cell. Signals from two or more different effector compounds of the invention expressed a cell or cells, can be separately resolved from other expressed effector compounds of the invention, by one or more features such as the location of the anchor site and the type of detectable indicator in each expressed effector compound. In certain aspects of the invention, anchor binding sequences, an indicator molecule sequence, and a sensor molecule sequence in an effector compound expressed in a cell or plurality of cells, may all be different from, all the same as, or some different from and some the same as: anchor binding sequences, an indicator molecule sequence, and a sensor molecule sequence, respectively, in a different effector compound expressed in the cell or plurality of cells. As used herein, the term “plurality” when used in reference to cells or effector compounds of the invention means “two or more”. In certain aspects of the invention 1, 4, 5, 6, 7, 8, 9, 10, 11, 12, 13, 14, 15, 16, 17, 18, 19, 20, 21, 22, 23, 24, 25, or more effector compounds may be expressed in a cell.

### Cells and Subjects

Some aspects of the invention include the use of multiplex imaging methods in one or more cells to image multiple activities, alterations, etc. within the one or more cells. A cell in which method of the invention may be may be used may be a prokaryotic or eukaryotic cell. In certain embodiments of the invention, a cell that can be imaged using methods of the invention may be a mammalian cell; including but not limited to a cell of a human, non-human primate, dog, cat, horse, cow, rodent, etc. In some embodiments of the invention, method of the invention may be used in a non-mammalian cell; including but not limited to an insect cell, an avian cell, a fish cell, a plant cell, etc. In some aspects of the invention, a cell that may be used is an artificial cell. Examples of a cell in which a multiplex imaging method of the invention may be used are non-excitable cells and excitable cells, the latter of which includes cells able to produce and respond to electrical signals. Examples of excitable cell types include, but are not limited, to neurons, muscles, cardiac cells, and secretory cells (such as pancreatic cells, adrenal medulla cells, pituitary cells, etc.).

Non-limiting examples of cells that may be used in methods of the invention include: neuronal cells, nervous system cells, cardiac cells, circulatory system cells, visual system cells, auditory system cells, secretory cells, endocrine cells, and muscle cells. In some embodiments of the invention, a cell used in conjunction with a spatial multiplex imaging method of the invention may be a healthy normal cell, which is not known to have a disease, disorder, or abnormal condition. In some embodiments, a cell used in conjunction with a spatial multiplex imaging method of the invention may be an abnormal cell, for example, a cell that has been diagnosed as having a disorder, disease, or condition, including, but not limited to a degenerative cell, a neurological disease-bearing cell, a cell model of a disease or condition, an injured cell, etc. In some embodiments of the invention, a cell may be a control cell.

Spatial multiplex imaging methods and compounds of the invention may be used in one or more cells from culture, in solution, obtained from subjects, and/or in a subject (in vivo cells). Spatial multiplex imaging methods of the invention may be used in cultured cells, cultured tissues (e.g., brain slice preparations, etc.), and in living subjects, etc. As used herein, the term “subject” may refer to a human, non-human primate, cow, horse, pig, sheep, goat, dog, cat, bird, rodent, insect, or other suitable vertebrate or invertebrate organism. In certain embodiments of the invention, a subject is a mammal and in certain embodiments of the invention a subject is a human.

### Controls and Candidate Compound Testing and Screening

One or more processes and/or conditions in one or more cells may be imaged using embodiments of spatial multiplex methods of the invention and methods of the invention can also be used to determine the presence of absence of change in one or more process or condition in one or more imaged cells. Thus, some aspects of the invention provide methods of determining the presence or absence of one or more changes or modulations in a cell. Some embodiments of the invention may include use spatial multiplex imaging methods of the invention to identify effects of candidate compounds on cells, tissues, and subjects. Results obtained by imaging in a cell using a spatial multiplex method of the invention can be advantageously compared to a control. In some embodiments of the invention imaging of one or more cells using a spatial multiplex method of the invention may be performed in a cell or plurality of cells and used to test the effect of candidate compounds on the cell or plurality of cells. A “test” cell may be a cell in which the activity in a cell may be tested or assayed. Results obtained using assays and tests of a test cell using a method of the invention may be compared results obtained from the assays and tests performed in other test cells or assays and/or may compared to a control value.

As used herein a control value may be a predetermined value, which can take a variety of forms. It can be a single cut-off value, such as a median or mean. It can be established based upon comparative groups, such as cells or tissues that have been imaged under similar conditions using a spatial multiplex imaging method of the invention, but are not contacted with a candidate compound with which the test cell is contacted and imaged. Another example of comparative groups may include cells or tissues that have a disorder or condition and groups without the disorder or condition. Another comparative group may be cells from a group with a family history of a disease or condition and cells from a group without such a family history. A predetermined value can be arranged, for example, where a tested population is divided equally (or unequally) into groups based on results of testing. Those skilled in the art are able to select appropriate control groups and values for use in comparative methods of the invention.

As a non-limiting example of use of a spatial multiplex method of the invention to assess the presence or absence of a change in a cell as a means to identify a candidate compound; the physiology and/or activity of a cell may be imaged using a spatial multiplex method of the invention in culture or in a subject and the cell may then be contacted the candidate compound and re-imaged using the spatial multiplex method of the invention. Any change in the physiology and/or activity in the imaged cell may indicate an effect of the candidate compound on the cell. In one embodiment of the invention, methods of the invention may be used to image the physiology and/or activity in one or more test cells before and after the one or more test cells are contacted with a candidate compound and the before and after spatial multiplex imaging results can be compared to determine whether or not contact with the candidate compound resulted in a change in one or more activities in the one or more test cells. In another embodiment of the invention, imaging of one or more activities in one or more test cells using a spatial multiplex method of the invention may be performed after contacting the one or more test cells with a candidate compound and the imaging results can be compared to control values for imaging of the one or more activities to determine whether or not a change in the one or more activities occurred in the one or more contacted test cells.

A cell, tissue, and/or subject that include a cell imaged with a spatial multiplex method of the invention may be monitored for the presence or absence of a change that occurs in the test conditions versus the control conditions.

As a non-limiting example, in a cell, a change in one or more activities may include, but is not limited to a change gene regulation activity in the cell, etc. Art-known methods can be used to assess the effect or impact one or more activities imaged using spatial multiplex methods of the invention, with or without additional contact with a candidate compound.

### Delivery of Effector Compounds

Delivery of an effector compound that localizes to and binds to a predetermined anchor site in a cell and comprises a sensor and an indicator, for example, a fluorescent molecule may be done using art-known delivery means. In some embodiments of the invention an effector compound that comprises a fluorescent molecule is included in a fusion protein. It is well known in the art how to prepare and utilize fusion proteins. In certain embodiments of the invention, a fusion protein can be used to deliver an effector compound of the invention to a predetermined location in a cell and may also be used to target an effector compound of the invention to specific cells or to a specific region in a cell, such as but not limited to, the cell nucleus. For example, an effector compound of the invention may comprise a TALE sequence and a sensor molecule that includes a fluorescent molecule and thus be used to target specific predetermined genomic locations in a cell nucleus. Targeting and identification of suitable targeting sequences for delivery of a sensor molecule and indicator molecule (such as but not limited to a fluorescent indicator molecule) to a predetermined cell or cell region can be performed using art-known procedures. (See for example, Saniana, N. E. et al., 2012 Nature Protocols 7, 171-192, the content of which is incorporated herein by reference.)

It is an aspect of the invention to provide an effector compound that comprises a sensor molecule and indicator signal molecule of the invention that is non-toxic, or substantially non-toxic in cells in which it is expressed. In some embodiments of the invention, an effector compound is genetically introduced into a cell and reagents and methods are provided for genetically targeted expression of the effector compound. Genetic targeting can be used to deliver an effector compound of the invention to specific cell types, to specific cell subtypes, to specific spatial regions within an organism, and to sub-cellular regions within a cell. Genetic targeting also relates to the control of the amount of an effector compound of the invention that is expressed, and the timing of the expression.

Some embodiments of the invention include a reagent for genetically targeted expression of an effector compound of the invention wherein the reagent comprises a vector that contains the gene for the effector compound that comprises the effector molecule, a sensor molecule and an indicator molecule (such as but not limited to: a fluorescent molecule) or a functional variant thereof.

As used herein, the term “vector” refers to a nucleic acid molecule capable of transporting between different genetic environments another nucleic acid to which it has been operatively linked. The term “vector” also refers to a virus or organism that is capable of transporting the nucleic acid molecule. One type of vector is an episome, i.e., a nucleic acid molecule capable of extra-chromosomal replication. Some useful vectors are those capable of autonomous replication and/or expression of nucleic acids to which they are linked. Vectors capable of directing the expression of genes to which they are operatively linked are referred to herein as “expression vectors”. In some aspects of the invention polypeptides of the invention may be expressed using prokaryotic or eukaryotic expression systems including bacterial and mammalian expression systems. Non-limiting examples of useful vectors include: viruses such as lentiviruses, retroviruses, adenoviruses, and phages. Vectors useful in some methods of the invention can genetically insert an effector compound of the invention into dividing and non-dividing cells and can insert an effector compound of the invention into cells that are in vivo, in vitro, or ex vivo cells.

Vectors useful in methods of the invention may include additional sequences including, but not limited to one or more signal sequences and/or promoter sequences, or a combination thereof. Expression vectors and methods of their use are well known in the art. Non-limiting examples of suitable expression vectors and methods for their use are provided herein. Vectors useful in certain methods of the invention can genetically insert one or more effector compounds of the invention thereof into dividing and non-dividing cells and can insert an effector polypeptide or variant thereof to cells that are in vivo, in vitro, or ex vivo cells.

The invention includes, in some aspects, functional variants of effector molecules for use in spatial multiplex compounds and methods of the invention. A function of an effector molecule variant can be tested by cloning the gene encoding the altered polypeptide into a bacterial or mammalian expression vector, introducing the vector into an appropriate host cell, expressing the altered polypeptide, and testing for a functional capability of the polypeptide as disclosed herein. Additional methods for generating fusion proteins and recombinant polypeptides are known in the art may include use of prokaryotic and eukaryotic expression systems including but not limited to bacterial and mammalian expression systems.

In certain embodiments of the invention, a vector may be a lentivirus comprising the gene for an effector compound of the invention. A lentivirus is a non-limiting example of a vector that may be used to create stable cell line. The term “cell line” as used herein is an established cell culture that will continue to proliferate given the appropriate medium.

Promoters that may be used in methods and vectors of the invention include, but are not limited to, cell-specific promoters or general promoters. Methods for selecting and using cell-specific promoters and general promoters are well known in the art. A non-limiting example of a general purpose promoter that allows expression of an effector compound of the invention in a wide variety of cell types—thus a promoter for a gene that is widely expressed in a variety of cell types, for example a “housekeeping gene” can be used to express an effector compound of the invention in a variety of cell types. Non-limiting examples of general promoters are provided elsewhere herein and suitable alternative promoters are well known in the art.

The present invention in some aspects, includes one or more methods of preparing and using an effector compound of the invention, identifying polypeptide and/or polynucleotide sequences useful in spatial multiplex imaging methods of the invention;

expressing in cells and membranes polypeptides encoded by the prepared polynucleotide sequences; using the expressed polypeptides for spatial multiplex imaging of one or more activities within the cell. The present invention enables spatial multiplex imaging in biological systems and an effector compound that comprises a fluorescent molecule of the invention and their use, have broad-ranging applications for drug screening, treatments, and research applications, some of which are describe herein.

### Effector Compound Variants

Effector compound variants can be prepared and used in embodiments of methods of the invention. For example, although not intended to be limiting, effector compound variants can be identified based on sequence similarity to the sequence of the effector compound but with one or more modifications in the effector compound polypeptide sequence or its encoding polynucleotide sequence. A variant of an effector compound may include a variant of one or more of the effector molecule's components, which are referred to herein as: the sensor molecule, the indicator molecule, and the effector molecule. Effector compounds and variants thereof of the invention may be identified, tested for function, and used in imaging methods of the invention according to procedures described herein. As used herein, the term “parent” when used in the context of a variant effector compound or component of the invention means the effector compound or component of which the variant effector compound is the variant. Based on the teaching provided herein regarding effector compounds, functional variants of effector compounds or component molecules that have sufficient amino acid sequence similarity/identity to a parent effector compound or component molecule sequence, respectively, and have at least a portion of the function of the parent effector compound or component molecule in methods of the invention, can be prepared and used in imaging methods of the invention.

As used herein, the term “identity” refers to the degree of relatedness or similarity between two or more polypeptide sequences [or polynucleotide (nucleic acid) sequences]. Sequence identify may be determined by the alignment and match between the sequences using standard methods. The percentage is obtained as the percentage of identical amino acids in two or more sequences taking account of gaps and other sequence features. The identity between polypeptide sequences can be determined by means of art-known procedures. Algorithms and programs are available and routinely used by those in the art to determine identity between polypeptide sequences and to determine identity between nucleic acid sequences. Non-limiting examples of programs and algorithms include BLASTP, BLASTN and FASTA (Altschul et al., NCB NLM NIH Bethesda Md. 20894; Altschul et al., 1990), Online BLAST programs from the National Library of Medicine are available, for example, at blast.ncbi.nlm.nih.gov/Blast.cgi.

The presence of functionality of a variant, for example the ability to be used in imaging methods of the invention, can be determined using assay and testing methods described herein. Functional variants of an effector compound can be used in imaging methods described herein. It will be understood that the level of sequence identity with a effector compound of the invention, and the level of functionality with respect to localization, activity sensing, imaging, etc. can be characteristics used to identify effector compound variants and other effector compounds using the teaching provided herein in conjunction with standard procedures for sequence alignment, comparisons, and knowledge of sequence modifications in the protein arts. A functional effector compound variant of the invention will have at least a portion of the functionality of the effector compound of which it is a variant. As used herein, the terms “functional” and “functionality” used in reference to an effector compound means the ability to be expressed in the appropriate location, sense activity, indicate sensed activities, and perform in imaging methods of the invention as described herein. A functional variant need not have an identical level of function as its parent effector compound, but will have at least a portion of the functionality of the parent effector compound and in some instances may have a level of function equivalent to, or higher than that of its parent effector compound when used in methods of the invention.

A variant of an effector compound may comprise the polypeptide sequence of the parent effector compound, with one or more sequence modifications. It will be understood that a modified sensor polypeptide, a modified indicator polypeptide and/or a modified effector polypeptide may be referred to herein as components of a “modified effector compound polypeptide”. A modified effector compound may include at least one modification in one or more of: a sensor polypeptide, an indicator polypeptide, and an effector polypeptide that are part of the effector compound. As used herein the term “modified” or “modification” in reference to a polypeptide sequence refers to a change such one or more of an insertion, deletion, or substitution of 1, 2, 3, 4, 5, 6, 7, 8, 9, 10, or more amino acids in the sequence as compared to the unmodified effector compound sequence of the invention. In some embodiments of the invention a modified effector compound sequence may include of 1, 2, 3, 4, 5, 6, 7, 8, 9, 10, or more amino acid substitutions in an effector compound polypeptide sequence of the invention.

An effector compound variant of the invention may include one or more sequence modifications of the parent effector compound and the resulting effector compound variant can be tested using methods described herein for characteristics including, but not limited to: expression, cell localization, imaging, etc. An example of a modification includes, but is not limited to one or more conservative amino acid substitutions, which will produce molecules having functional characteristics similar to those of the molecule from which such modifications are made. Conservative amino acid substitutions are substitutions that do not result in a significant change in the activity or tertiary structure of a selected polypeptide or protein. Such substitutions typically involve replacing a selected amino acid residue with a different residue having similar physico-chemical properties. For example, substitution of Glu for Asp is considered a conservative substitution because both are similarly sized, negatively charged amino acids. Groupings of amino acids by physico-chemical properties are known to those of skill in the art. The following groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). Effector compound variants that include modifications from their parent effector compound, such as, but not limited to one, two, three, four, or more conservative amino acid substitutions can be identified and tested for characteristics including, but not limited to: expression, cell localization, imaging characteristics, etc., using methods disclosed herein.

An effector compound variant may include modifications that result in an amino acid sequence having at least 80%, 85%, 90%, 95%, 96%, 97%, 98%, 99%, or 100% identity with the amino acid sequence of the effector compound of which it is a variant. In addition, a variant of an individual component of an effector compound such as an indicator polypeptide, a sensor polypeptide, or an effector polypeptide may include modifications that result in an amino acid sequence having at least 80%, 85%, 90%, 95%, 96%, 97%, 98%, 99%, or 100% identity with the amino acid sequence of the indicator polypeptide, sensor polypeptide, or effector polypeptide, respectively, of which it is a variant. Sequence identity can be determined using standard techniques known in the art. In some embodiments of the invention, an effector compound variant may be shorter or longer than sequence of which it is a variant.

It will be understood that different combinations of components of effector compounds of the invention may be prepared, tested, and used as set forth herein. For example, two effector compounds may have one or more of a different indicator (e.g., fluorophore), sensor, and effector molecules. Different combinations of indicator polypeptides, sensor polypeptides, and effector polypeptides are envisioned in certain aspects of effector compounds and methods of the invention.

Another aspect of the invention provides nucleic acid sequences that code for an effector compound of the invention or a variant thereof. It would be understood by a person of skill in the art that an effector compound polypeptide sequence of the invention and variants thereof, can be encoded (coded for) by various nucleic acids. Each amino acid in the protein is represented by one or more sets of three nucleic acids (codons). Because many amino acids are represented by more than one codon, there is not a unique nucleic acid sequence that codes for a given protein. It is well understood by those of skill in the art how to make a nucleic acid that can encode a polypeptide when the amino acid sequence of the polypeptide is known. A nucleic acid sequence that codes for a polypeptide or protein is the “gene” of that polypeptide or protein. A gene can be RNA, DNA, or other polynucleotide than will code for the polypeptide or protein. As used herein the term “nucleic acid” is used interchangeably with the term “polynucleotide”.

It is understood in the art that the codon systems in different organisms can be slightly different, and that therefore where the expression of a given protein from a given organism is desired, the nucleic acid sequence can be modified for expression within that organism.

Thus, in some embodiments, an effector compound polypeptide or variant thereof of the invention is encoded by a mammalian-codon-optimized nucleic acid sequence, which may in some embodiments be a human-codon optimized nucleic acid sequence. An aspect of the invention provides a nucleic acid sequence that codes for an effector compound polypeptide or variant thereof, that is optimized for expression with a mammalian cell. In some embodiments of the invention, a nucleic acid that encodes an effector compound polypeptide or variant thereof includes a nucleic acid sequence optimized for expression in a human cell.

An effector compound of the invention may be delivered to a cell or a plurality of cells in a composition. In some aspects a composition may be a pharmaceutical composition. Pharmaceutical compositions used in the embodiments of the invention preferably are sterile and contain an effective amount of an effector compound to permit expression and imaging in a cell, plurality of cells and/or a subject.

The amount of an effector compound of the invention to successfully image in a cell, plurality of cells and/or a subject can be chosen in accordance with different parameters, in particular in accordance with the mode of administration. Other factors may include the desired period of imaging, the number of cells to be imaged, etc.

### Compositions and Delivery

In general a composition that includes one or more effector compounds of the invention includes the one or more effector compounds and may also include a carrier, which may be pharmaceutically-acceptable carrier. Pharmaceutically acceptable carriers are well known to the skilled artisan and may be selected and utilized using routine methods. As used herein, a pharmaceutically-acceptable carrier means a non-toxic material that does not interfere with the effectiveness of the activity of the active ingredients, e.g., the binding, sensing, and imaging function of the expressed effector compound in the cell and/or subject to which it is administered. Carriers and pharmaceutically acceptable carriers may include diluents, fillers, salts, buffers, stabilizers, solubilizers and other materials that are well-known in the art. Exemplary pharmaceutically acceptable carriers are described in U.S. Pat. No. 5,211,657 and others are known by those skilled in the art. Preparations for administration of an effector compound of the invention may include preparation and/or use of sterile aqueous or non-aqueous solutions, suspensions, and emulsions.

In some embodiments of the invention, an effector compound of the invention may be administered directly to a tissue. For example, though not intended to be limiting, an effector compound of the invention may be administered to the brain or other tissue in a subject by direct injection, intravenous delivery, or other suitable art-known means.

### Additional Effector Compound Imaging Methods

Methods of the invention, in some aspects include methods of using a genetically encoded activity reporter (Effector compound) to assess one or more of an activity, alteration, process, condition or other physiological characteristic of a cell in which the effector compound is expressed. In certain aspects, methods of the invention may include methods to assess an activity, process, or condition in a cell. In some aspects of the invention, assessment methods may include expressing an effector compound of the invention in a cell. The expressed effector compound will comprise a sensor molecule, an indicator molecule and an effector molecule. The cell is then imaged and the imaging permits an assessment of a process or condition in the cell, based at least in part on the imaging of the indicator polypeptide of sensor molecule in the cell. Thus, if it is of interest to assess gene regulation in a cell, an effector compound or variant thereof of the invention that includes a sensor molecule selected because it provides information on gene regulation, may be expressed in a cell and imaging performed to detect the indicator in response to gene regulation in the cell.

Certain aspects of the invention also permit use of an effector compound or variant thereof of the invention to assess an effect of candidate agent on a process, activity, or condition in a cell. In such methods an effector compound or variant thereof may be expressed in a cell, wherein the effector compound includes a sensor molecule, an indicator molecule, and an effector molecule. The expressed effector compound itself, or another region of a cell or tissue can then be contacted with a candidate agent; and the cell can be imaged using methods of the invention to assess one or more of a process, condition, or activity in the cell or an impact on the cell from the contact of another cell or tissue with the agent, based at least in part on the imaging. The results of the imaging can be compared to a control assessment of the process, activity, or condition; and if there is a difference between the process or condition as assessed in the cell as compared to the control assessment of the process, activity, or condition it indicates that the candidate compound modulated or changed one or more of the process, activity, and condition in the cell. In some aspects of the invention a control assessment is an assessment in a cell in which the same type of effector compound is expressed but that was not contacted with the candidate compound.

Certain aspects of the invention include methods and compounds with which to identify a chromosome anchor site that may be used in imaging methods of the invention. The method may include identifying a chromosome binding site, for example a TALE chromosome binding site that has a polynucleotide sequence that: includes more than 20 repetitive chromosome binding sequences, each having a sequence of from 18 to 22 by in length. In some aspects of the invention the length of a chromosome binding sequence that binds to an anchor sequence may be 17, 18, 19, 20, 21, 22, 23 base pairs in length. A chromosome anchor site that is useful in methods of the invention may, but is not required to, begin with the polynucleotide thymine.

In some aspects of the invention assessing whether a candidate anchor site is suitable for use in methods of the invention, includes determining that the identified anchor site is at least one of chromosome specific and at least 5000 by away from an active gene. Although for optimizing brightness of the signal in imaging methods of the invention, it may be beneficial to include at least 20 repetitive chromosome binding sequences, it will be understood that in certain embodiments of the invention fewer than 20 repetitive chromosome binding sequences may be included. In situations when fewer than 20 repeats are included, the brightness of a cluster may be reduced, necessitating extending the exposure time for imaging. In certain aspects of the invention, inclusion of 15, 16, 17, 18, 19, 20 or more repetitive chromosome binding sequences can be used to optimize temporal resolutions in dynamic monitoring methods of the invention.

As a non-limiting example, in some aspects of the invention; a TALE chromosome binding site polynucleotide sequence includes 20 repetitive chromosome binding sequences, each of the binding sequences is 20 by in length, and the first nucleotide of the sequences is optionally thymine, and in certain aspects of the invention the first nucleotide of the sequence may be guanine (G) or adenine (A).

### EXAMPLES

### Example 1

### Methods:

### Bioinformatics Design in DNA Binding Sites:

TALE binding sites were searched in hg20 (human) and mm 10 (mouse) genome database, respectively. Each binding site contained more than 20 repetitive binding sequences, with each sequence was 20 bp in length and started with a thymine. The selected binding sites were assured to be chromosome specific, and at least 5000 by away from the active genes. The selected binding sites allow at least 20 copies of TALE-fused sensors clustering with sufficient brightness for imaging. The selected sites, because they do not overlap with active genes, minimize the chance of expressed TALE proteins regulating the gene expression.

### Construct Cloning:

The genes encoded the GCaMP6f, GEICS1.3, AKAR4, and ICUE3 proteins fused to nuclear localization signal (NLS) and corresponding antibody tag were subcloned into the modified pCI vector backbone (Promega) using NheI and EcoRI sites. The pCI vector was modified by removing BsaI site from bla gene using QuickChange mutagenesis (Agilent Technologies). For neuronal expression CMV promoter was swapped with CAG promoter. TALE binding proteins were subcloned to the 5′ end of the sensor genes, using the standard golden-gate assembly method, forming 20 mer TALE proteins fused to the sensors via a (GGSGGSGGT)×3 linker sequence, which is set forth herein as SEQ ID NO: 1 GGSGGSGGTGGSGGSGGTGGSGGSGGT. The genes encoded the FRET sensor proteins were digested from plasmids Addgene 61619 and 61622, respectively (see Depry C, et al., 2011 Mol Biosyst. 2011 January; 7(1):52-8 and DiPilato L M & Zhang J. 2009 Mol Biosyst. August; 5(8):832-7, the content of each of which are incorporated herein by reference. The GCaMP6f molecules were as described in Chen T. W. et al., 2013 Nature. July 18; 499(7458): 295-300. CKAR sequence as described in Violin J D, et al., 2003 J Cell Biol June 9. 161(5):899-909.

### Transfection in Cultured Cells:

HEK cells were transfected with the TransIT®-293 transfection reagent (Minis). Cells were plated in a 24-well plate, transfected with 50 ng plasmids per well after 18-24 hours plating, and imaged 24 hours after transfection. Mouse neurons were transfected with the calcium phosphate transfection kit (Invitrogen). Neurons were plated in a 24-well plate, transfected 96 hours after plating with 1500 ng plasmids per well and were imaged at least 48 hours after transfection. For co-transfection in neurons, two plasmids were mixed as 1:1 ratio, maintaining a total of 1500 ng plasmids per well. For HEK experiments, NucBlue Life cell stain and ReadyProbes reagent (Life Technologies) were added to stain the nucleus. Mfn1-null MEF cells (ATCC® CRL-2992™) were plated in a 24-well glass-bottom plate with 50-70% cell confluency. Plasmids were then transfected with a mixture of 200 ng plasmid and 0.6 mL Trans-IT X2 (Mirus) per well. Transfected cells were incubated at 37° C. for 48-72 hours to establish the expression.

### DNA Fluorescent In-Situ Hybridization (FISH):

The Histology FISH Accessory Kit (Agilent, K5799) was used with several modifications in the manufacturer's protocol. Cells were fixed by 4% formaldehyde for 10 minutes and left in 70% ethanol at 4° C. overnight. Afterwards cells were permeabilized with 0.5% Triton100 for 10 minutes on a 37° C. hotplate, followed by 2×SSC at 75° C. for 10 minutes and an ethanol wash. The chromosome DNAs in cells were denatured together with the FISH probe (Agilent Sure FISH, G101067R) at 66° C. for 10 minutes, followed by 1.5 hr hybridization at 45° C. After the hybridization, the sample was rinsed in Dako stringent wash buffer for 10 minutes at 63° C., followed by water washes at 37° C. for 1 minute, and sequentially 70%, 85%, and 100% ethanol washes at room temperature, each lasting 1 minute. Dako fluorescent mounting medium was added to minimize photo-bleaching.

### Live Cells Imaging:

An inverted epi-fluorescence Nikon Ti Eclipse equipped with SPECTRA X light engine (LumenCor), Zyla5.5 camera (Andor), 100× 1.45 NA oil objective lens (Nikon), 20× 0.75 NA objective lens (Nikon), and standard CFP-YFP FRET filter sets (Semrock) were used to image the fluorescent patterns. FRET imaging typically lasts for 10 min. A 50 μM forskolin (Sigma, pre-dissolved in DMSO) was added at around 2-3 mins by pipetting to monitor the cell response, when steady baselines in both YFP and CFP channels were achieved. For inomycin treatment of HEK293 cells 20 mM ionomycin dissolved in DMSO (Sigma Aldrich, St. Louis, Mo.) was added at around 2 minutes by pipetting to monitor the cell response, when steady baseline in GFP channel Imaging was conducted in darkness at room temperature

### Results

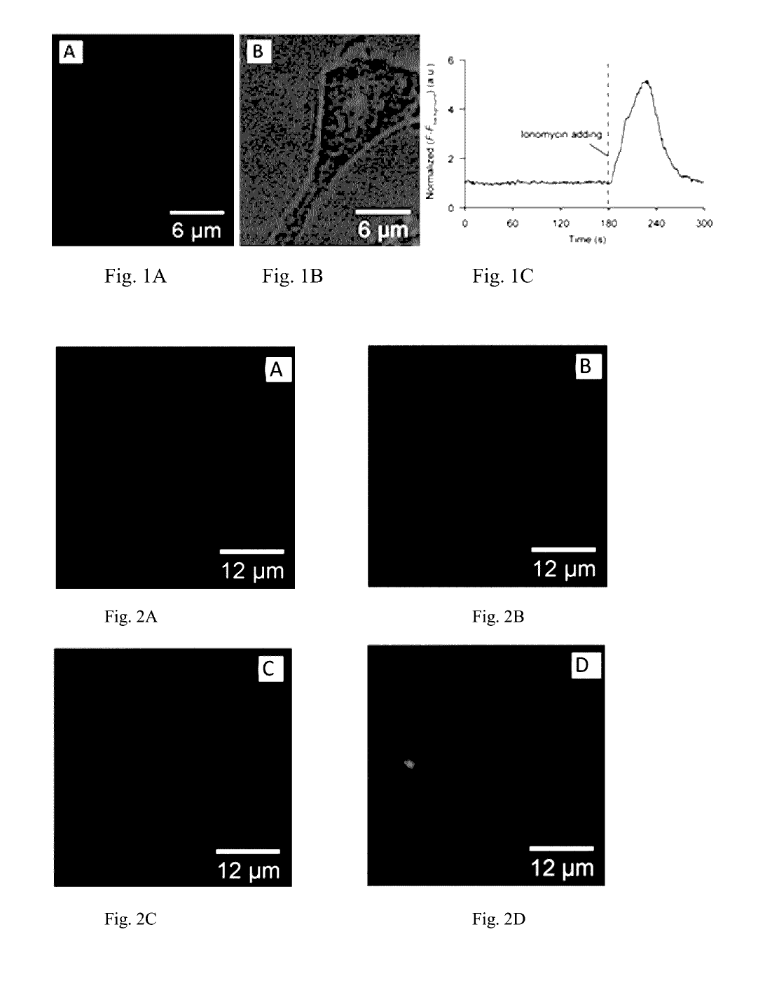

The results from experiments performed in HEK293 cells are shown in FIG. 1A-C. The experiments demonstrated sensor clustering and illustrated responses to an ionomycin treatment in HEK cells. The TALE sequence used in experiments with results illustrated in FIGS. 1 and 2 was: tggagcgattcgtgtagtat (SEQ ID NO: 2). FIG. 1A shows Gcamp6f sensor clustering. FIG. 1B shows an overlay image of the bright field and the Gcamp sensor clustering. Both images were taken with with 100×, 1.45 NA. Scale bar: 6 μm. FIG. 1C is a graph that shows how the Gcamp6f sensor cluster responded to an ionomycin treatment to the cell. The fluorescence values were normalized after subtracting the extracellular background. The results indicated that the sensor cluster was present in the HEK293 cells and that the treatment with ionomycin resulted in an incrase in fluorescence by the Gcamp6f sensor cluster.

FIG. 2A-D shows DNA FISH results in HEK cells from experiments performed to assure the binding specificity of TALE proteins. FIG. 2A shows Gcamp6f sensors (green); FIG. 2B shows DNA FISH probes (red); FIG. 2C shows NucBlue nuclear stain; FIG. 2D shows an overlay image of FIG. 2A, FIG. 2B, and FIG. 2C. All images were taken with 20×, 0.45 NA. Scale bar: 12 μm.

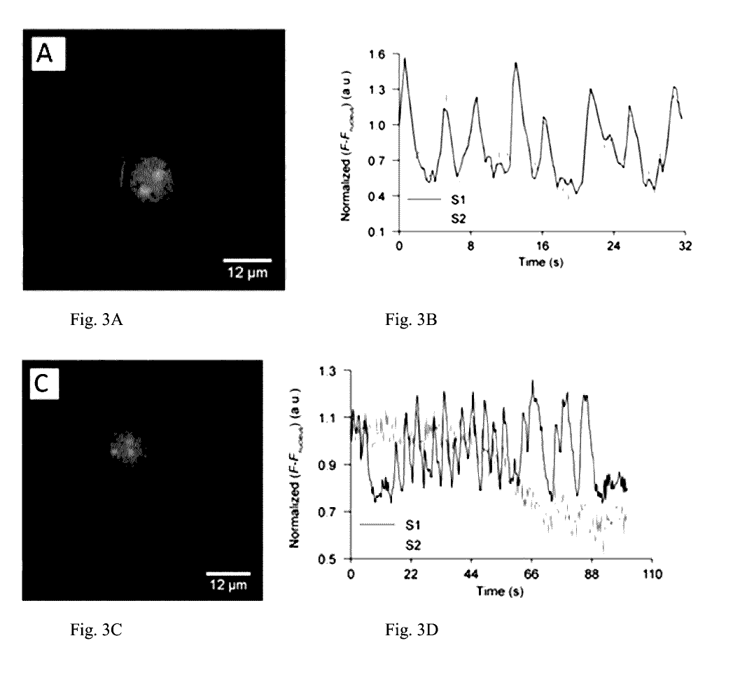

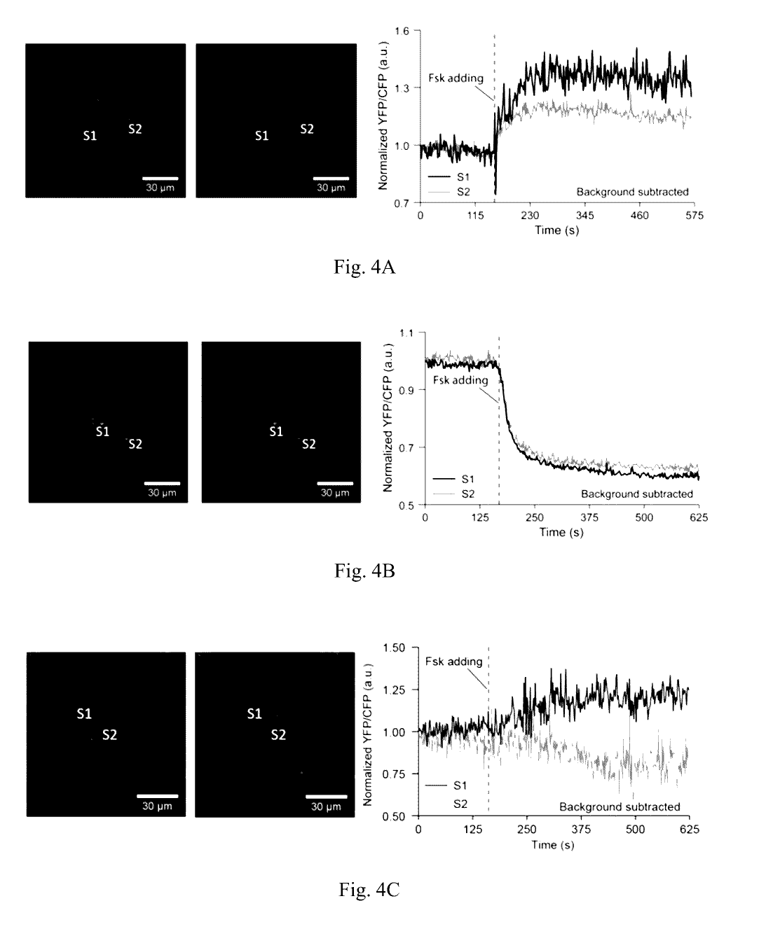

FIG. 3A-D shows results of Hippocampus mouse neuron experiments. The TALE sequences used in experiments with results shown in FIGS. 3 and 4 were: tgctacgactaggtatacct (SEQ ID NO: 3); and tcggacggcccttcgccatc (SEQ ID NO: 4). The images show typical sensor clustering and responses in hippocampus mouse neurons. FIG. 3A shows one neuron single-transfected with Gcamp6f sensors. Typically two sensor clusters were expressed in the cell (bright field). FIG. 3B shows results demonstrating that a typical neuron single-transfected with Gcamp6f, all clusters show identical fluorescence patterns as neuron spikes; FIG. 3C shows results of one neuron co-transfected with Gcamp6f and GEICS1.3 sensors. Typically three sensor clusters were expressed in the cell (bright field); FIG. 3D shows results demonstrating that in a typical neuron co-transfected with Gcamp6f and GEICS 1.3, sensor clusters showed different fluorescence patterns as neuron spikes. The images in FIG. 3A and FIG. 3C were imaged with 100× 1.45 NA. Scale bar: 12 μm. The fluorescence values in FIG. 3B and FIG. 3D were normalized after subtracting the background in cell nucleus.

FIG. 4A-C shows photomicroscopy images and graphs illustrating results obtained in MEF cells that expressed embodiments of FRET sensors of the invention. The results showed typical FRET sensor clustering and responses in mouse embryonic fibroblast (MEF) cells. FIG. 4A shows results from one MEF cell single-transfected with AKAR4 sensors with YFP/CFP FRET pairs. Sensor clusters were expressed in the cell, confirmed by the co-localization between YFP and CFP channels (left panel); a 50 μM FSK stimulus (added at around 2 min after start of recording) caused consistent increase of the fluorescence ratio, YFP/CFP. FIG. 4B shows results from another MEF cell single-transfected with ICUE3 sensors with YFP/CFP FRET pairs; a 50 μM FSK stimulus caused consistent decrease of the fluorescence ratio, YFP/CFP. FIG. 4C shows results from another MEF cell that was co-transfected with both AKAR4 and ICUE3 sensors with YFP/CFP FRET pairs; a 50 μM FSK stimulus caused a consistent increase of the fluorescence ratio YFP/CFP for AKAR4 and caused a consistent decrease of the fluorescence ratio YFP/CFP for ICUE3. The images were imaged with 20× 0.75 NA. Scale bar: 30 μm. The fluorescence values were normalized after subtracting the extracellular background.

### EQUIVALENTS

It is to be understood that the methods and compositions that have been described above are merely illustrative applications of the principles of the invention. Numerous modifications may be made by those skilled in the art without departing from the scope of the invention.

Although the invention has been described in detail for the purpose of illustration, it is understood that such detail is solely for that purpose and variations can be made by those skilled in the art without departing from the spirit and scope of the invention which is defined by the following claims.

The contents of all literature references, publications, patents, and published patent applications cited throughout this application are incorporated herein by reference in their entirety.

We claim:

1. A method of spatial multiplex imaging in a cell, the method comprising
expressing in a cell one, two, or more effector compounds each comprising: (a) an independently selected sensor molecule comprising an indicator molecule and (b) an independently selected effector molecule, wherein the effector molecule binds a predetermined anchor site in the cell, and when two or more effector molecules are expressed in the cell each binds a different predetermined anchor site in the cell; and wherein the sensor molecule comprises an independently selected indicator molecule; and
detecting the one, two or more independently selected indicator molecules in the cell.

2. The method of claim 1, wherein two or more effector compounds are expressed in the cell, and detecting comprises detecting each of the independently selected indicator molecules.

3. The method of claim 1, further comprising determining a cellular activity or signal indicated by the detection of the indicator molecule of the independently selected sensor molecules of the one, two, or more effector compounds.

4. The method of claim 1, wherein the anchor site comprises chromosomal DNA.

5. The method of claim 1, wherein the indicator molecule comprises a fluorescent polypeptide.

6. The method of claim 1, wherein the effector compound comprises a TALE polypeptide and sensor molecule comprising a fluorescent polypeptide indicator.

7. The method of claim 1, wherein the cell is a mammalian cell.

8. (canceled)

9. The method of claim 1, wherein the anchor site comprises a sequence of chromosomal DNA that specifically binds the effector molecule, the binding of the effector molecule is chromosome specific; and wherein the chromosomal DNA that specifically binds the effector molecule is present in no more than one chromosome of the cell and contains at least 5 consecutive repeats of the specific DNA sequence to which the effector molecule binds.

10. The method of claim 1, wherein the anchor site comprises a sequence of chromosomal DNA that specifically binds the effector molecule is present in no more than one chromosome of the cell, contains at least 6, 7, 8, 9, 10, 11, 12, 13, 14, 15, 20, 30, 40, 50 consecutive repeats of the specific DNA sequence to which the effector molecule binds, and contains no active genes.

11. (canceled)

12. A method of identifying a spatial multiplex imaging binding site, the method comprising:
(a) selecting a first effector compound comprising a first sensor molecule comprising a first indicator molecule and a first effector molecule that specifically binds a predetermined anchor site in a cell, wherein the anchor site comprises a sequence of chromosomal DNA that specifically binds the first effector molecule, the binding of the first effector molecule is chromosome specific; and the chromosomal DNA that specifically binds the first effector molecule is present in not more than one chromosome of the cell, and contains at least 5 consecutive repeats of the specific DNA sequence to which the first effector molecule binds;
(b) expressing in a cell the first effector compound; and
(c) detecting the first indicator molecule that is expressed in the cell.

13. The method of claim 12, further comprising
(d) selecting one or more additional independently selected effector compounds each comprising: (i) an independently selected sensor molecule comprising an independently selected indicator molecule and (ii) an independently selected effector molecule that specifically binds an anchor site in a cell, wherein the anchor site comprises a sequence of chromosomal DNA that specifically binds the independently selected effector molecule, the binding of the independently selected effector molecule is chromosome specific; and the chromosomal DNA that specifically binds the independently selected effector molecule is: present in not more than one chromosome of the cell, and contains at least five consecutive repeats of the specific DNA sequence to which the independently selected effector molecule binds;
(e) expressing in the cell of (b) the one or more additional independently selected effector compounds; and
(f) detecting the indicator molecule of the first effector compound and the indicator molecule of the one or more independently selected effector compounds expressed in the cell.

14. The method of claim 12, wherein the anchor site comprises a sequence of chromosomal DNA that specifically binds the effector molecule, the binding of the effector molecule is chromosome specific; and wherein the chromosomal DNA that specifically binds the effector molecule is present in one chromosome of the cell, contains at least 2, 3, 4, 5, 6, 7, 8, 9, 10, 11, 12, 13, 14, 15, 20, 30, 40, or 50 consecutive repeats of the specific DNA sequence to which the effector molecule binds, and contains no active genes.

15. The method of claim 12, wherein the indicator molecule is a fluorescent indicator polypeptide.

16. The method of claim 12, wherein the effector compound comprises a TALE polypeptide and sensor molecule comprising a fluorescent polypeptide indicator.

17. The method of claim 12, wherein the cell is a mammalian cell.

18. (canceled)

19. An effector compound, wherein the effector compound comprises: (a) a sensor molecule comprising an indicator molecule and (b) an effector molecule that when expressed in a cell, comprises a DNA-binding sequence that binds a predetermined chromosomal DNA anchor site in the cell, wherein the binding of the expressed effector molecule is chromosome specific; and wherein the chromosomal DNA that specifically binds the expressed effector molecule is present in no more than one chromosome type in the cell, contains at least 5, 6, 7, 8, 9, 10, 11, 12, 13, 14, 15, 20, 30, 40, or 50 consecutive repeats of the specific DNA sequence to which the expressed effector molecule binds, and contains no active genes.

20. The effector compound of claim 19, wherein the effector compound is in a composition comprising a carrier, optionally a pharmaceutically acceptable carrier.

21-22. (canceled)

23. The effector compound of claim 19, wherein the effector compound is a polypeptide molecule.

24. The effector compound of claim 19, wherein the effector compound is in a cell.

25. (canceled)

26. A vector comprising the effector compound of claim 19.

27-44. (canceled)


SEQUENCE LISTING (4 sequences)

SEQ ID NO: 1 - protein, 27 aa, Artificial Sequence
  Synthetic polypeptide
GGSGGSGGTGGSGGSGGTGGSGGSGGT

SEQ ID NO: 2 - DNA, 20 bp, Artificial Sequence
  Synthetic polynucleotide
tggagcgattcgtgtagtat

SEQ ID NO: 3 - DNA, 20 bp, Artificial Sequence
  Synthetid polynucleotide
tgctacgactaggtatacct

SEQ ID NO: 4 - DNA, 20 bp, Artificial Sequence
  Synthetic polynucleotide
tcggacggcccttcgccatc